(19) United States
(12) Patent Application Publication
(10) Pub. No.: US 2016/0242955 A1
(43) Pub. Date: Aug. 25, 2016

(54) Body Temperature Control System

(71) Applicant: ICETRON TECHNOLOGIES LTD., Yokneam (IL)

(72) Inventors: Roee Karmona, Kiryat Bialik (IL); Liran Tetro, Kiryat Motzkin (IL); Haim Ben Amram, Kiryat Motzkin (IL)

(21) Appl. No.: 14/917,323

(22) PCT Filed: Nov. 9, 2014
(86) PCT No.: PCT/IB2014/065904

§ 371 Date: Mar. 8, 2016

Related U.S. Application Data
(60) Provisional application No. 61/904,005, filed on Nov. 14, 2013.

(51) Int. Cl. A61F 7/00; A61F 7/02

(52) U.S. Cl. CPC A61F 7/007; A61F 7/02; A61F 2007/0034; A61F 2007/0075; A61F 2007/0078; A61F 2007/0086; A61F 2007/0096

(57) ABSTRACT

This invention relates to a body temperature control apparatus including a container comprising a heat absorbent substance; a heat sink in communication with the container; a thermal isolation element connected with the container; a plate supported on thermal isolation element and designed to contact the human skin; a thermoelectric module connected with the heat sink and with the plate; a temperature sensor connected with the plate; and a controller configured to electrically control the thermoelectric module and the temperature sensor.

### CROSS-REFERENCE TO RELATED PATENT APPLICATIONS

This patent application claims priority from and is related to U.S. Provisional Patent Application Ser. No. 61/904,005, filed Nov. 14, 2013, this U.S. Provisional Patent Application incorporated by reference in its entirety herein.

### FIELD OF THE INVENTION

The present invention is in the technical field of temperature control systems and more particularly to an electrical body temperature control system.

### BACKGROUND

The mammal body regulates its own temperature, but in cases that the body produces or absorbs more heat than it can dissipate, the temperature of the mammal rises. A good example can be physical exertion or a hot day.

There are several known devices for cooling the mammal internal temperature. Most of these devices are based on pre-stored thermal or chemical energy or evaporation-based clothing such as cold vests and ice packs.

### SUMMARY

According to a first aspect of the present invention there is provided a body temperature control apparatus comprising: a container comprising a heat absorbent substance; a heat sink in communication with the container; a thermal isolation element connected with the container; a plate supported on thermal isolation element and designed to contact the human skin; a thermoelectric module connected with the heat sink and with the plate; a temperature sensor connected with the plate; and a controller configured to electrically control the thermoelectric module and the temperature sensor.

The heat absorbent substance may be water.

The heat sink may be made from high thermal conductivity material.

The plate may be made from high thermal conductivity material.

The thermoelectric module may be a single-stage thermoelectric module or a multi-stage thermoelectric module.

The apparatus may further comprise a power source configured to power the controller.

The power source may be a battery.

The apparatus may further comprise at least one additional sensor, which may be selected from the group consisting of: GPS sensor, accelerometer sensors, motion sensor, UV radiation sensor and pedometer.

The controller may further control the at least one additional sensor.

The controller may further comprise memory and processing means configured to run at least one application.

The at least one application may be configured to process data from the at least one sensor.

The apparatus may further comprise user interface means.

The user interface means may comprise a display configured to display the processed data.

The display may comprise a touch screen.

The touch screen may be configured to receive user selections.

The user selections may comprise cooling or heating.

The user interface means may comprise one of a speaker and a buzzer.

The controller may further control the user interface means.

The apparatus may be configured to be attached to a human limb.

The attaching may comprise contacting the plate with the limb's pulse point.

### BRIEF DESCRIPTION OF THE DRAWINGS

For better understanding of the invention and to show how the same may be carried into effect, reference will now be made, purely by way of example, to the accompanying drawings.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 is a perspective view of a system according to the present invention;

FIG. 2 is a side view of a system according to the present invention;

FIG. 3 is a sliced perspective view of a system according to the present invention;

FIG. 4 is a sliced side view of a system according to the present invention; and

FIG. 5 is a schematic drawing of an exemplary control device according to the present invention.

### DETAILED DESCRIPTION OF PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

It will be appreciated by persons skilled in the art that the present invention is not limited to what has been particularly shown and described hereinabove. Rather the scope of the present invention is defined by the appended claims and includes combinations and sub-combinations of the various features described hereinabove as well as variations and modifications thereof which would occur to persons skilled in the art upon reading the foregoing description.

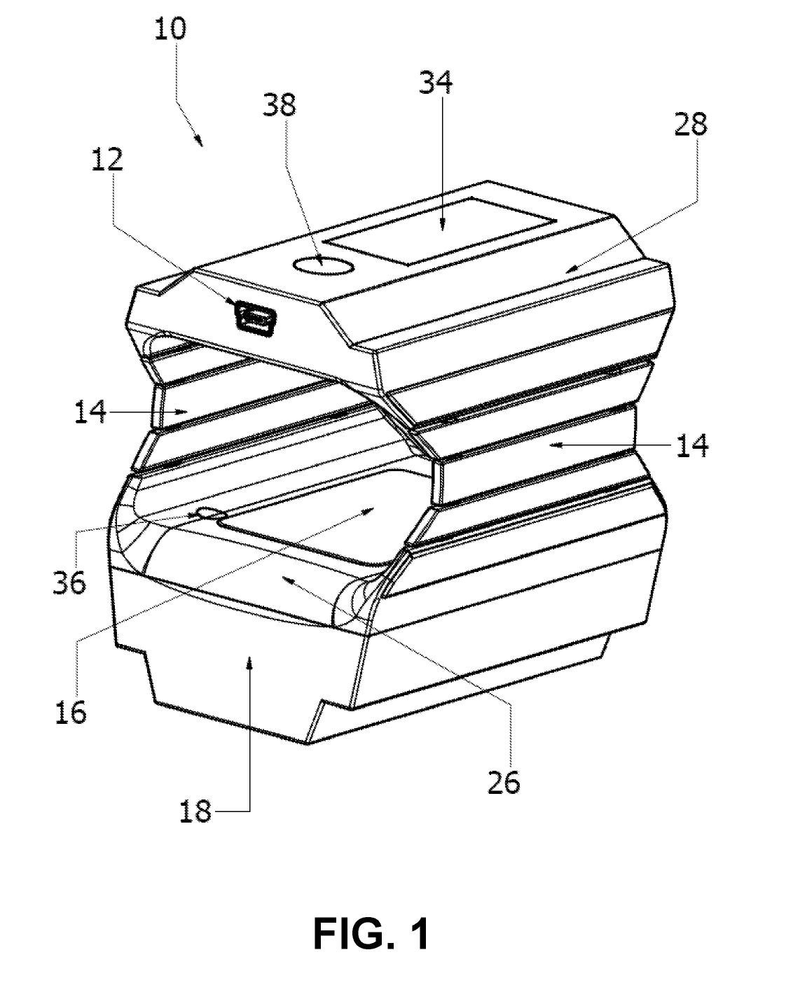

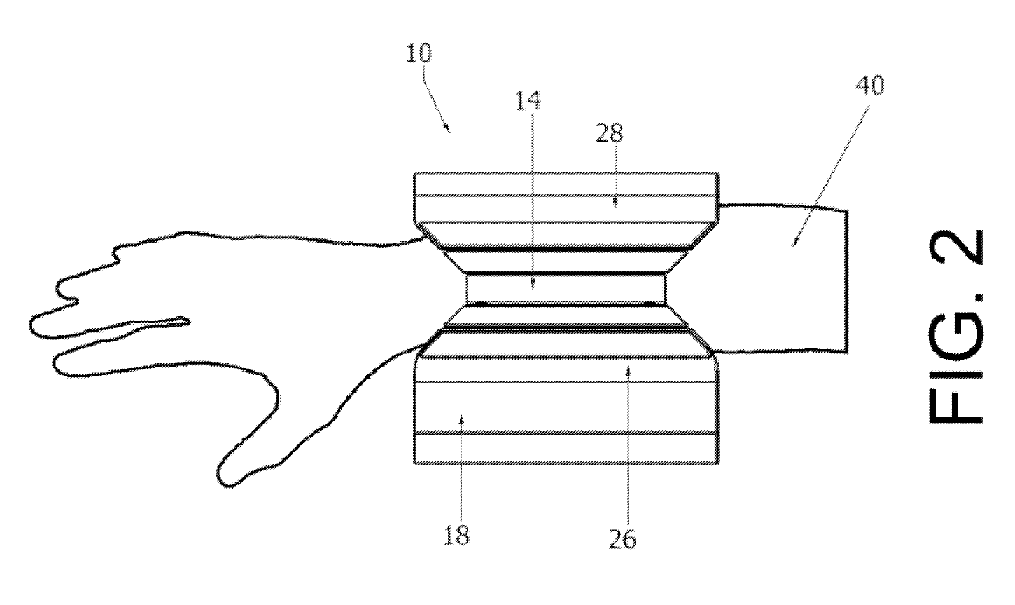

Referring now to the invention in more detail, FIGS. 1 and 2 show respectively perspective and side views of the device of the present invention in an assembled state.

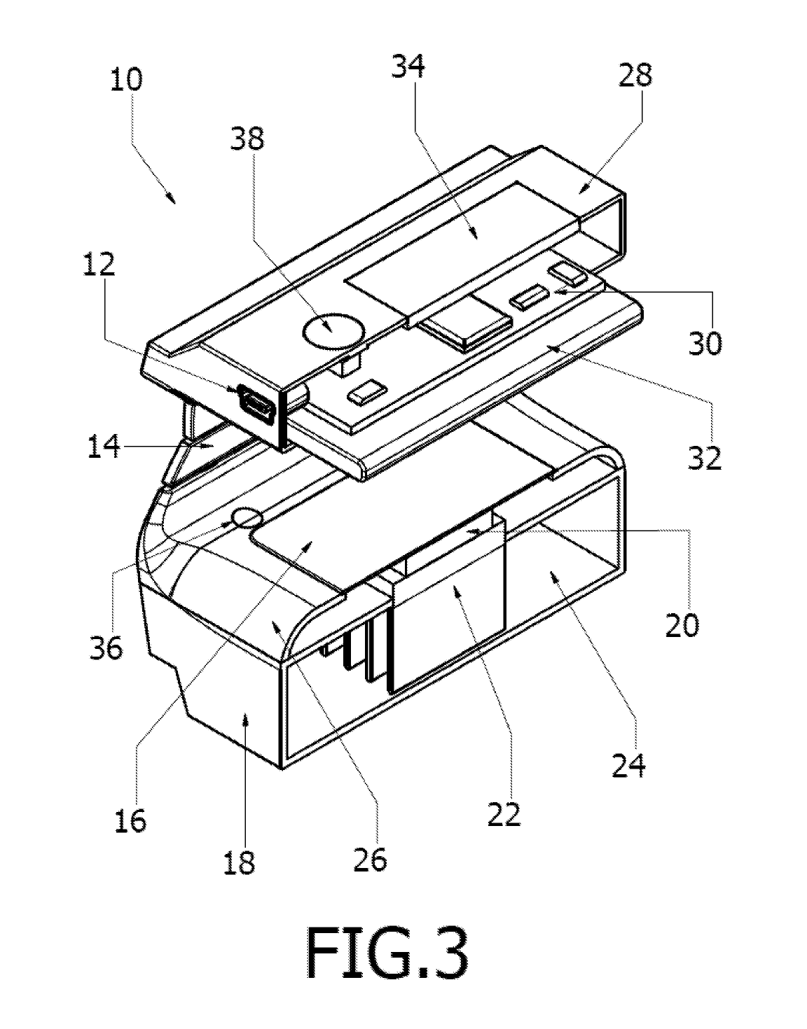

Temperature control system 10 includes:
A control housing 28, which contains various components as will be detailed in conjunction with FIG. 3;
A container 18 designed to be attached to an optional thermal isolation element 26, which is configured to fit the limb 40 and to provides thermal isolation between container 18 and limb 40. Container 18 is configured to contain a heat absorbent substance 24, which is preferably a highly absorbent substance such as water. The heat absorbent substance 24 is configured to absorb thermal energy from the heat sink 22. The container 18 is preferably made from a water resistant material (only if the heat absorbent material is liquid). The volume of liquid in the container is preferably between 30 and 200 cm³.
Elastic side bands 14 connecting between control housing 28 and thermal isolation element 26; The side bands 14 are designed to enable fitting of the temperature control system 10 to the limb 40;
An optional plate 16 supported on thermal isolation element 26 and designed to contact the human skin. In a preferred embodiment the plate 16 contacts limb 40, preferably close to the limb's pulse point, to have maximum effectiveness. Plate 16 is preferably made from high thermal conductivity material such as copper or aluminum, or any other high thermal conductivity material. Plate 16 is mounted onto the thermal isolation element 26, preferably attached thereto by adhesive.
Heat sink 22, preferably made from high thermal conductivity material such as copper or aluminum, or any other high thermal conductivity material. Heat sink 22 is configured to absorb thermal energy from the thermoelectric module 20. The heat sink 22 is preferably mounted in container 18. The connection between heat sink 22 and container 18 is preferably made with a sealer, to create a sealed container. The thermal isolation element 26 provides thermal isolation between container 18 and limb 40.
A temperature sensor 36 mounted on plate 16.

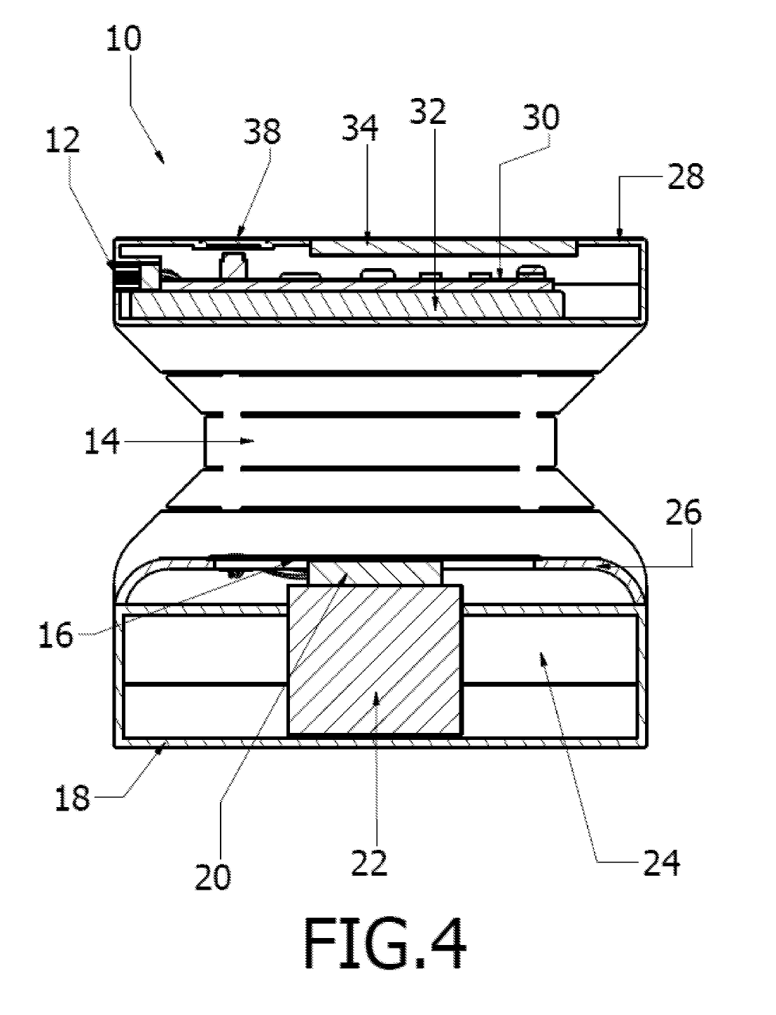

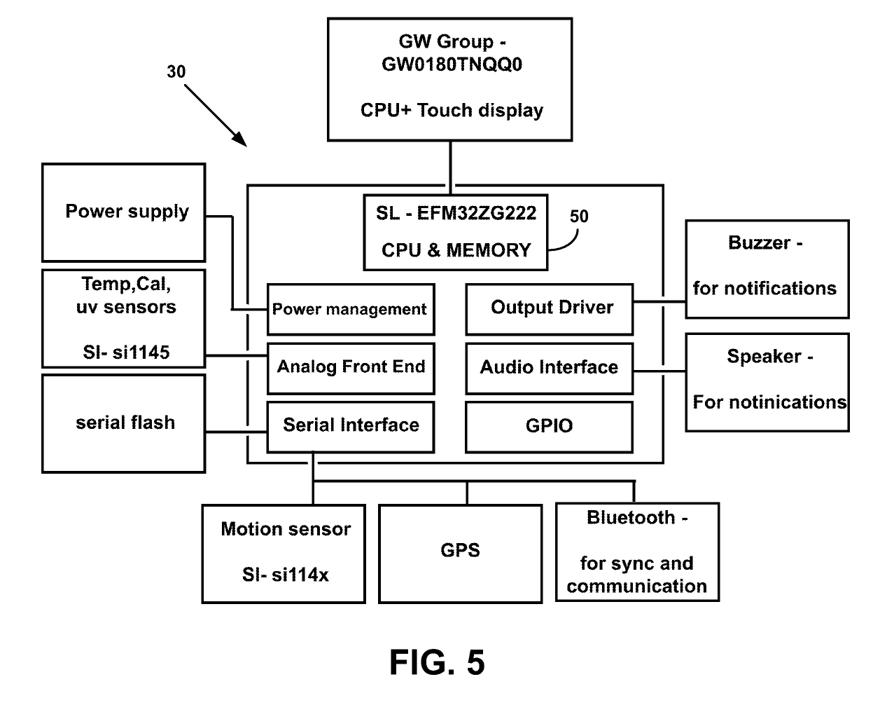

In further detail, referring now to FIGS. 3 and 4 that represent a sliced view of the temperature control system 10, control housing 28 contains:
A control device 30, preferably a printed circuit board, containing suitable electronic components for controlling and operating the temperature control system 10, as will be described in detail in conjunction with FIG. 5;
A thermoelectric module 20 connected to the control device 30. The thermoelectric module 20 is preferably a typical single-stage thermoelectric module, such as Peltier Cooler Model 9502/065/012 M available from Ferrotec (htts://www.ferrotec.com/) and can also be a multi-stage module, such as Peltier Cooler Model 9530/119/045 B, also available from Ferrotec. Preferably, the connection wires of thermoelectric module 20 go through side band 14 and thermal isolation element 26. Preferably, the thermoelectric module 20 is also attached to the plate 16 by thermal grease. Thermoelectric module 20 can also be in direct contact with limb 40. Preferably the thermoelectric module 20 is attached to heat sink 22 by thermal grease.
A control switch 38 connected to control device 30. Control switch 38 can enable or disable the operation of temperature control system 10.
A power source 32, such as a battery, preferably chargeable, provided to power the temperature control system 10. Power source 32 is typically connected with the control device 30. Alternatively the temperature control system 10 may be powered by an external power source;
Optional USB port 12, which can be any USB connector, may be provided for charging the optional battery 32 and for importing and exporting data. Alternatively USB port 12 may be replaced with any other suitable connector such as, for example, AUX or Thunderbolt;
Optional touch screen 34 may be provided for controlling and operating the temperature control system 10, e.g. for selecting cooling or warming mode and for displaying various data and information;

In operation, when cooling is desired, the control device 30, powered by power source 32, operates the thermoelectric module 20, which uses the Peltier effect to cool the plate 16, i.e. when DC current flows through the thermoelectric module 20, it brings heat from one side to the other, so that one side gets cooler while the other gets hotter. Temperature sensor 36 measures the temperature of plate 16 (or of the limb 40) and transfers the temperature data to the control device 30 for controlling the operation intensity of thermoelectric module 20 accordingly, e.g. to maintain plate 16's temperature in the range of 15 to 20 degrees Celsius.

The system of the present invention may alternatively serve as a heating system by reversing the current direction because the direction of heating and cooling is determined by the polarity of the applied voltage.

In operation, when reversing the current polarity, the control device 30, powered by power source 32, operates the thermoelectric module 20, which uses the Peltier effect to heat the plate 16, i.e. when DC current flows through the thermoelectric module 20, it transfers cold from one side to the other, so that one side gets hotter while the other gets cooler. Temperature sensor 36 measures the temperature of plate 16 (or of the limb 40) and transfers the temperature data to the control device 30 for controlling the operation intensity of thermoelectric module 20 accordingly, e.g. to maintain plate 16's temperature in the range of 37 to 50 degrees Celsius.

According to embodiments of the invention, various additional sensors can be added to the device of the present invention. Preferably sensors that need to come in contact with limb 40 such as pulse and temperature sensors, etc. Other types of sensors that do not need to contact limb 40 such as GPS and accelerometer sensors, motion sensor, UV radiation sensor, pedometer etc. can be mounted on control device 30.

FIG. 5 is a schematic drawing of the control device 30 comprising microcontroller, such as EFM32ZG222, available from Silicon Labs (www.silabs.com), a display, preferably touch screen for bi-directional communication with the device 10 and a number of interfaces to auxiliary modules such as a power management module for controlling the power supply, an analog front-end for controlling analog sensor such as temperature, calories and UV sensors such as SI1145 Digital UV Index, available from Grobotronics (www.grobotronics.com), a serial interface for controlling e.g. serial flash memory, a motion sensor such as Si114x, available from Silicon Labs (www.silabs.com), GPS or Bluetooth, an output driver for controlling e.g. a buzzer for notification, an audio interface for controlling e.g. a speaker for notification and a General-purpose input/output (GPIO) for controlling other optional auxiliary devices.

According to embodiments of the invention, various applications can be hosted on the CPU for retrieving sensors data, processing it and displaying it to the user on the touch screen. Such data may include battery usage, time, distance calculator, etc.

According to embodiments of the invention, the various parts of cooling system 10 may be interconnected using a variety of methods, such as, for example, ultrasonic soldering, adhesive such as epoxy or silicon, injection techniques such as insert molding and mechanical grippers such as snap fasteners or screws.

In an experiment done we used a 3.7V 1500 mAh battery and a container holding 60 cm³ of water. We achieved approximately 100 minutes of continuous work.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains. For example, the cooling system 10 could take many different forms. Thus the scope of the invention should be limited only by the following claims.

1. A body temperature control apparatus comprising:
a sealed container comprising a heat absorbent substance;
a heat sink in connection with said sealed container;
a thermoelectric module connected with the heat sink and with a plate;
a temperature sensor connected with said plate; and
a controller configured to electrically control said thermoelectric module based on said temperature sensor.

2. (canceled)

3. The apparatus of claim 1, wherein said heat sink is made from high thermal conductivity material.

4. (canceled)

5. (canceled)

6. The apparatus of claim 1, further comprising a thermal isolation element connected with said sealed container.

7. The apparatus of claim 1, further comprising a plate designed to contact the human skin.

8. The apparatus of claim 7, wherein said plate is made from high thermal conductivity material.

9. The apparatus of claim 1, further comprising a power source configured to power said controller.

10. (canceled)

11. The apparatus of claim 1, further comprising at least one additional sensor and wherein said controller further controls said at least one additional sensor.

12. The apparatus of claim 11, wherein said at least one additional sensor is selected from the group consisting of: GPS sensor, accelerometer sensors, motion sensor, UV radiation sensor and pedometer.

13. (canceled)

14. The apparatus of claim 12, wherein said controller further comprises memory and processing means configured to run at least one application, said at least one application is configured to process data from said at least one sensor.

15. (canceled)

16. The apparatus of claim 14, further comprising user interface means.

17. The apparatus of claim 16, wherein said user interface means comprise a display configured to display said processed data.

18. The apparatus of claim 17, wherein said display comprises a touch screen configured to receive user selections comprising cooling or heating.

19. (canceled)

20. (canceled)

21. The apparatus of claim 16, wherein said user interface means comprise one of a speaker and a buzzer.

22. (canceled)

23. The apparatus of claim 1, configured to be attached to a human limb.

24. The apparatus of claim 23, wherein said attaching comprises contacting the plate with the limb's pulse point.
